(19) United States
(12) Patent Application Publication
(10) Pub. No.: US 2019/0091124 A1
(43) Pub. Date: Mar. 28, 2019

(54) AQUEOUS N-ACYL AMINO ACID SOLUTIONS

(71) Applicant: MINASOLVE GERMANY GMBH, Leuna (DE)

(72) Inventors: Markus NAHRWOLD, Minden (DE); Nadia KONATÉ, Magdeburg (DE)

(21) Appl. No.: 16/083,436

(22) PCT Filed: Feb. 21, 2017
(86) PCT No.: PCT/EP2017/053855

§ 371 Date: Sep. 7, 2018

(30) Foreign Application Priority Data
Mar. 8, 2016 (DE) 10 2016 104 205.6

(51) Int. Cl. A61K 8/44; A61Q 5/00; A61Q 19/10; A61Q 15/00; A61Q 19/00; A61K 8/06

(52) U.S. Cl. CPC A61K 8/442; A61Q 5/006; A61K 8/062; A61Q 15/00; A61Q 19/00; A61Q 19/10

(57) ABSTRACT

A method for producing aqueous N-acyl amino acid solutions does not require any solid to be isolated and yields solutions that have a low impurity content. The corresponding aqueous N-acyl amino acid solutions can be used in cosmetic and pharmaceutical preparations. The solutions can include an alkaline metal or alkaline earth metal salt of capryloyl glycinic acid and/or undecenoyl glycinic acid.

The invention relates to a method for producing aqueous N-acyl amino acid solutions. The method does not require any solid to be isolated and yields solutions that have a low impurity content. The invention further relates to the corresponding aqueous N-acyl amino acid solutions, as well as to the use thereof in cosmetic and pharmaceutical preparations. N-acyl amino acids (“lipoamino acids”) are used in cosmetic and pharmaceutical products. Examples of representatives of this class of substances include capryloyl glycine (I) and undecylenoyl glycine (II). These are the N-acyl amides of the amino acid glycine and of the fatty acids caprylic acid (=octanoic acid) and undecenoic acid, respectively.

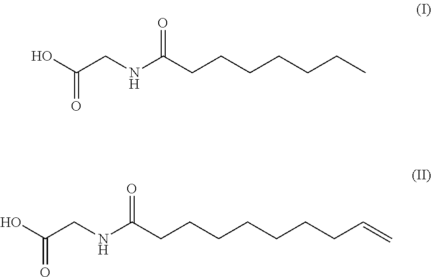 [SMILES: CCCCCCCC(=O)NCC(=O)O ; C=CCCCCCCCC(=O)NCC(=O)O]

Capryloyl glycine (I) is marketed by the company MINASOLVE under the trade name “Caprocine”. Capryloyl glycine shows broad-spectrum antimicrobial activity against bacteria, fungi and yeasts. The substance can be used as a preservative and as a deodorant. In addition, capryloyl glycine effectively inhibits the growth of the bacterium Propionibacterium acnes, which plays a major role in the development of acne. The substance also inhibits the growth of the yeast Malassezia furfur (Pityrosporum ovale), which contributes to dandruff formation on the scalp. Additionally, capryloyl glycine inhibits the enzyme 5-alpha-reductase, which positively influences sebum production in the skin. Capryloyl glycine is thus able to reduce sebum production in the skin and consequently diminish the tendency to develop dandruff and acne. N-acyl amino acids are generally low in toxicity and well-tolerated on the skin. The use thereof in cosmetic and pharmaceutical products is therefore of interest.

N-acyl amino acids are generally solids, which are only moderately soluble in water. For incorporation in cosmetic or pharmaceutical formulations, N-acyl amino acids are therefore neutralized with bases and thus converted into their water-soluble carboxylic acid salts. To this end, use is often made of highly corrosive alkaline substances such as sodium hydroxide or potassium hydroxide. The strong bases are not compatible with all of the raw materials that are used in cosmetic or pharmaceutical preparations. In such cases, the N-acyl amino acids must then be neutralized in a separate stirring vessel. As an alternative, N-acyl amino acids are dissolved in hot water or in another lipophilic solvent. In this type of application, the free N-acyl amino acids cause a decrease of the pH in the formulation. This can also negatively impact the stability of a formulation. There are disadvantages associated with the manipulation of solids in general. As a rule, it is necessary to ensure, with long stirring times, that the solid has completely dissolved in the selected solvent. Furthermore, dust may be produced, which for example leads to irritations of the skin, eyes and/or airways. A contamination of the work environment is also possible. As a whole, the use of solid N-acyl amino acids is associated with increased expenditures in terms of time, energy, materials and consequently costs.

On the industrial scale, N-acyl amino acids are synthesized by condensing amino acids with carboxylic acid chlorides or anhydrides in alkaline aqueous solution (Schotten-Baumann reaction). Examples of such syntheses are described in the literature, for example in FR 2771632 A1 (SEPPIC, Priority: 01.12.1997) and WO 2006/010590 A1 (Sinerga, Priority: 30.07.2004). As a result of chemical reaction, an alkaline raw product solution is formed, which in addition to the N-acyl amino acid salt also contains at least one inorganic salt such as table salt (NaCl) in an equimolar quantity. This non-purified aqueous N-acyl amino acid solution can in principle be used as-is as a raw material for cosmetic or pharmaceutical products. However, a disadvantage lies in the possibility that the inorganic salts contained in the solution interact with other components of a formulation. For example, such salts can affect the rheology of an emulsion or gel. Also disadvantageous is the presence of free alkyl carboxylic acids in the raw product solution. Alkyl carboxylic acids are by-products of Schotten-Baumann synthesis that arise through hydrolysis of the activated carboxylic acid. Depending on the length of their carbon chains, alkyl carboxylic acids can act as skin irritants and have a characteristic unpleasant odor. The staining that sometimes occurs is another disadvantage associated with the direct use of non-purified N-acyl amino acid solutions in cosmetics.

As a rule, the reaction mixtures obtained in the manner described above are therefore reconditioned. N-acyl amino acids contained in the mixture are precipitated by acidification and the precipitated solid is optionally crystallized using a suitable organic (co-)solvent. Organic impurities, for example non-converted free amino acids, are simultaneously removed with this purification step. However, the isolation and drying of the purified solid entails an additional expenditure in terms of work time and material, which in turn leads to higher production costs.

The object of the invention is the production of concentrated aqueous N-acyl amino acid solutions, which are obtained directly and without the isolation of solids from a reaction mixture. The aqueous concentrate should contain the N-acyl amino acid in a high degree of purity, whereas the by-products and waste products should be contained in minimum concentration. The aqueous solution should furthermore be chemically and physically stable within a wide temperature range, and be as odorless and colorless as possible.

Surprisingly, the object was achieved by the synthesis method described as follows:
The alkaline raw product solution from a Schotten-Baumann reaction is acidified in the presence of an organic solvent. The solvent is chosen from substances that are only miscible with water to a limited extent, but that also form an azeotropic mixture with water. In the course of acidification, the N-acyl amino acid transfers to the organic solvent phase.
Inorganic salts and free amino acids are removed by washing the organic product solution with water.
The organic product solution is extracted with an aqueous solution of a base. In this process, the N-acyl amino acid, as a water-soluble salt, re-converts to the aqueous solution.
After renewed phase separation, the solvent residues contained in the aqueous product solution are removed by azeotropic distillation. Other by-products volatile with steam can be removed by further distillation of water. This applies, for example, to the free alkyl carboxylic acids arising as hydrolysis products during the Schotten-Baumann reaction. To this end, the pH must be adjusted to a neutral range of ca. 6-7 during the distillation. As a rule, N-acyl amino acids are stronger acids than the simply alkyl-substituted fatty acids. The fatty acids are thus in equilibrium at a neutral pH, partly as steam-volatile free carboxylic acids, whereas the N-acyl amino acids are preferably present as non-volatile salts.
A final adjustment of the pH to ≥6.5 stabilizes the N-acyl amino acid as a carboxylic acid salt in the aqueous solution.

The solutions obtained according to the invention are advantageous in many respects over known state-of-the-art solids or saline solutions: As liquids they can be easily metered and incorporated in liquid mixtures. In the process, they either cause no or only slight pH changes. The solutions are essentially free of inorganic salts and therefore do not interact negatively with salt-sensitive substances such as polymeric thickeners or salt-thickened surfactant gels. The solutions furthermore contain only minute quantities of free alkyl carboxylic acids, which positively influences their odor and skin tolerance. Moreover, the synthesis of the solutions obtained according to the invention is economical because solid/liquid separation is not required.

Any amino-carboxylic acid (amino acid) can be used as source material for synthesizing the N-acyl amino acid. In this process, chiral amino acids can be used as enantiomerically pure or diastereomerically pure compounds, and also as mixtures of different stereoisomers. Additional functional groups that may be present within the amino acids are protected by suitable protecting groups. Short peptides having up to 6 amino acids can also be used. The amino acid component is preferably selected from the group consisting of alanine, arginine, asparagine, aspartic acid, cysteine, glutamine, glutamic acid, glycine, glycylglycine, histidine, isoleucine, leucine, lysine, methionine, phenylalanine, dihydroxyphenylalanine (DOPA), proline, serine, threonine, tryptophan, tyrosine, valine, 3-aminopropanoic acid (beta-alanine) and 4-aminobutyric acid (gamma-aminobutyric acid, GABA). Particular preference is given to selecting the amino acid component from glycine and glycylglycine.

The fatty acid component is selected from any aliphatic fatty acid having a chain length of 6 to 22 carbon atoms, preferably 6 to 12 carbon atoms. The fatty acid can be saturated, mono-or polyunsaturated. It can contain either one or two carboxylic acid functions. The N-acyl amino acid preferably contains at least one of the following fatty acid parts: hexanoyl (caproyl), heptanoyl (oenanthoyl), octanoyl (capryloyl), nonanoyl (perlagonyl), decanoyl (caprinoyl), undecylenoyl, dodecanoyl (lauryl), tetradecanoyl (myristyl), hexadecanoyl (palmitoyl), heptadecanoyl (margarinoyl), octadecanoyl (stearyl), eicosanoyl (arachinoyl), docosanoyl (behenoyl), hexadecenoyl (palmitoleinoyl), octadecenoyl (oleyl), eicosenoyl (gadoloyl), docosenoyl (erucyl), octadecadienoyl (linoloyl), octadecatrienoyl (linolenoyl), eicosatetraenoyl (arachidoyl). Particular preference is given to the N-acyl amino acid containing one of octanoyl (capryloyl) and undecylenoyl fatty acid parts.

For synthesizing the N-acyl amino acid, the carboxylic acid can be activated as an acid halide, as a mixed anhydride or as a symmetrical anhydride. The carboxylic acid is preferably activated as an acid chloride.

Any acid having an acidity higher than that of the synthesized N-acyl amino acid can be used for lowering the pH of the raw product solution. Preference is given to using inorganic acids and/or acid salts thereof such as hydrochloric acid, sulfuric acid, sodium bisulfate, potassium bisulfate, phosphoric acid, monosodium phosphate, disodium phosphate, monopotassium phosphate and/or dipotassium phosphate. The adjusted pH is ≤7. The pH is preferably between 0 and 5, particularly preferably between 0.5 and 3.0.

As an organic solvent for the aqueous reconditioning of the N-acyl amino acid solution, use can be made of any organic solvents that are immiscible or not completely miscible with water. Either individual solvents or solvent mixtures can be used. Preference is given to using esters (e.g., ethyl acetate, butyl acetate, isopropyl acetate, isobutyl acetate), ethers (e.g., tert-butyl methyl ether, diisopropyl ether, 2-methyltetrahydrofuran), aromatic hydrocarbons (e.g., toluene, xylene), aliphatic hydrocarbons (e.g., heptane, cyclohexane, methylcyclohexane), ketones (e.g., ethyl methyl ketone, isobutyl methyl ketone), halogenated hydrocarbons (e.g., dichloromethane, dichloroethane, chloroform) and/or alcohols (e.g., n-butanol, isobutanol, amyl alcohol). Particular preference is given to using ethyl acetate, toluene, 2-methyltetrahydrofuran and/or tert-butyl methyl ether.

The phase separations during aqueous reconditioning can take place at any temperature at which both phases are present as liquids. This is typically the case at temperatures of −20° C. to +100° C. The phase separation is preferably carried out at +10° C. to +80° C., particularly preferably at +30° C. to +60° C. The phase separation can also take place under overpressure.

The azeotropic distillation for removing excess organic solvent can take place at any pressure. The distillation can be carried out at normal pressure (1013 mbar±50 mbar) and/or in a vacuum and/or under overpressure.

Any alkaline metal or alkaline earth metal base can be used as a base during the Schotten-Baumann reaction and for converting the N-acyl amino acid to the aqueous product solution. Preference is given to using sodium hydroxide, potassium hydroxide, sodium carbonate, potassium carbonate, sodium bicarbonate and/or potassium bicarbonate. The bases used can be used either as solids or as aqueous solutions.

The pH of the N-acyl amino acid solution synthesized according to the invention can be between 5 and 14. The end product solution is preferably adjusted to a pH between 6.5 and 8.5, inclusive.

The concentration of the N-acyl amino acid in the solutions produced according to the invention can be between 1% and 90%, preferably 10-50%, particularly preferably 20-40%. The specified values are understood as mass percentages based on the free N-acyl amino acid. The N-acyl amino acid is thus present mainly as a salt with the base used for neutralization. However, a portion of the N-acyl amino acid may be present as free carboxylic acid, depending on the pH.

The invention also relates to aqueous N-acyl amino acid solutions produced according to the method described. The solutions according to the invention are clear, stable and homogeneous within a broad temperature range. A crystallization of the solid components at low temperatures is just as unlikely to occur as a chemical decomposition of the components at higher temperatures. The solutions according to the invention are preferably physically and chemically stable in a temperature range of −20° C. to +80° C. The aqueous solutions according to the invention are typically self-preserving and therefore stable with regard to colonization or decomposition by microorganisms. As a rule, it is thus unnecessary to use additional preservatives to protect the solutions according to the invention.

If required, the solutions according to the invention can contain further additives. Examples of such include preservatives, cosmetic solvents, surface-active substances (surf actants/emulsifiers) and/or water-soluble polyvalent alcohols.

The solutions according to the invention can be used as raw materials in cosmetics and/or in personal hygiene products and/or in pharmaceutical preparations. They are thus able to perform functions that generally help achieve positive effects on age-induced and/or environmentally-induced skin or hair changes. They can be used as preservatives, deodorants, anti-acne agents, anti-dandruff agents, skin lightening agents, enzyme inhibitors and/or pH regulators, for example. Other possible applications include, but are not limited to, use as conditioners, foam boosters, (co-)emulsifiers and/or (co-)surfactants, enzyme inhibitors, light and UV protection, as well as for regulating sebum production and/or for influencing rheology or viscosity.

Using the solution according to the invention, it is also possible to synthesize a product, in particular a cosmetic and/or pharmaceutical and/or dermatological and/or hygienic product, containing a solution as explained above. In other words, the solution according to the invention is used in cosmetic and/or pharmaceutical and/or dermatological and/or hygienic products. Specifically, the terms “hygienic preparation” or “hygienic product” are understood to mean household or cleaning products as well as aromatic substance preparations.

The solutions according to the invention can be added to the cosmetic and/or pharmaceutical and/or dermatological and/or hygienic product at any time during production, e.g., during the production of an aqueous phase or at the end of the production process.

The solutions according to the invention can also be used in a product, particularly in a cosmetic or dermatological product, containing the solutions according to the invention, wherein said product contains 0.05-10.0 wt % of at least one N-acyl amino acid. The product con-cerned can occur in any form, in particular as:
a. solution,
b. suspension,
c. emulsion,
d. gel,
e. ointment,
f. paste,
g. powder,
h. solid in pieces or in a block,
i. foam,
j. formulation system based on microencapsulation, liposomes, or similar microscopic structures.
k. combinations of forms a-j

### EXAMPLES/EXPERIMENTAL PART

### Example 1—Production of an Aqueous Capryloyl Glycine Solution

Glycine (50 g) and sodium hydroxide (51 g) are dissolved in water (250 mL). Octanoyl chloride (87 g) is measured and added. MTBE (500 mL) and 36% hydrochloric acid (50 g) are added, successively, to the alkaline solution. The phases are separated, and the organic phase is washed with water. The organic phase is then mixed with water (600 mL) and the pH value of the mixture is adjusted to pH 6.5 with sodium hydroxide. The organic phase is separated. Excess organic solvent and water are distilled from the aqueous phase at normal pressure until a concentration of 30 wt % capryloyl glycine, based on the free acid, is reached. The pH value of the aqueous solution is then adjusted to pH 7.5-8.5 with sodium bicarbonate. The solution contains <0.1% sodium chloride and <0.5% octanoic acid.

The 30% aqueous capryloyl glycine solution fulfills the A criteria of a preservative efficacy test according to ISO 11930. This solution is therefore protected against microbiological contamination. An additional preservative is not required.

### Example 2—Anti-Dandruff Shampoo

| Phase | Raw Material | INCI Name | % |
|---|---|---|---|
| A | Water | Aqua | 50.0 |
| | TEGO Betain F 50⁽¹⁾ | Cocamidopropyl Betaine | 4.5 |
| | Texapon NSO UP⁽²⁾ | Sodium Laureth Sulfate | 35.0 |
| | Plantacare 1200 UP⁽²⁾ | Lauryl Glucoside | 3.0 |
| B | Solution according to | Water (and) Capryloyl Glycine | 3.0 |
| | Example 1 | (and) Sodium Bicarbonate | |
| C | Glucamate VLT | PEG-120 Methyl Glucose | 0.7 |
| | Thickener⁽⁴⁾ | Trioleate (and) Propanediol | |
| D | Perfume Orchidée | Perfume | 0.2 |
| | Blanche⁽⁵⁾ | | |
| | EasySafe 9010⁽³⁾ | Phenoxyethanol (and) | 1.0 |
| | | Ethylhexylglycerin | |
| | D&C Green #5 (1%)⁽⁶⁾ | Aqua (and) Green No. 5 | 0.6 |
| E | Table Salt | Sodium Chloride | 2.0 |
| F | Citric Acid (50%) | Aqua (and) Citric Acid | pH 5.5 |

Raw material suppliers:
⁽¹⁾Evonik,
⁽²⁾BASF,
⁽³⁾Minasolve,
⁽⁴⁾Lubrizol,
⁽⁵⁾SFA,
⁽⁶⁾Orco

The raw materials of Phase A are added into waterby stirring. The solution according to the invention is added (Phase B). The thickener (Phase C) is mixed in, with vigorous stirring. The ingredients of Phase D are added successively, followed by table salt (Phase E). The stirring of the mixture is continued until the solid is completely dissolved. Lastly, the pH is adjusted (Phase F). The shampoo fulfills the A criteria of a preservative efficacy test according to 11930.

### Example 3—Facial Cleanser

| Phase | Raw Material | INCI Name | % |
|---|---|---|---|
| A | Water | Water | 74.9 |
| | Methocel 40-202 PCG⁽¹⁾ | Hydroxypropyl | 0.2 |
| | | Methylcellulose | |
| B | Pentiol Green+⁽²⁾ | Pentylene Glycol | 2.0 |
| | Xanthan Gum PC⁽³⁾ | Xanthan Gum | 0.5 |
| C | Solution according to | Water (and) Capryloyl | 3.0 |
| | Example 1 | Glycine (and) Sodium | |
| | | Bicarbonate | |
| | 25% Caustic Soda | Water (and) Sodium | 0.1 |
| | | Hydroxide | |
| D | Plantacare 1200 UP⁽⁴⁾ | Lauryl Glucoside | 5.0 |
| | Plantapon ACG HC⁽⁴⁾ | Sodium Cocoyl Glutamate | 5.0 |
| | Rewoteric AMC⁽⁵⁾ | Sodium Cocoamphoacetate | 4.0 |
| E | Lamesoft PO 65⁽⁴⁾ | Coco-Glucoside (and) | 3.0 |
| | | Glyceryl Oleate | |
| | Glycerin | Glycerin | 2.0 |
| | Perfume Bamboo & | Perfume | 0.2 |
| | Lemongrass⁽⁶⁾ | | |
| | D&C Green # 5 (1%)⁽⁷⁾ | Aqua (and) Green No. 5 | 0.1 |
| F | Citric Acid (50%) | Aqua (and) Citric Acid | pH 5.5 |

Raw material suppliers:
⁽¹⁾Dow;
⁽²⁾Minasolve;
⁽³⁾Jungbunzlauer;
⁽⁴⁾BASF;
⁽⁵⁾Evonik;
⁽⁶⁾Bell;
⁽⁷⁾Orco

Methylcellulose is added into water, with stirring, at room temperature. The pH should not exceed 7.5 during this process (Phase A). Pentiol Green+ and Xanthan Gum are mixed (Phase B), and the mixture is added to Phase A. The mixture is stirred until it becomes homogeneous. The solution according to Example 1 is added, followed by a little caustic soda (Phase C). The mixture is continuously stirred for 15-20 min until fully thickened. The surfactants of Phase D are added, with stirring, in the order specified. Then the raw materials of Phase E are added successively, with stirring, in the order specified. Lastly, the pH is adjusted. The product fulfills the A criteria of a preservative efficacy test according to ISO 11930.

### Example 4—Roll-On Deodorant

| Phase | Raw Material | INCI Name | % |
|---|---|---|---|
| A | Water | Aqua | 71.9 |
| | Carbopol Ultrez 20 | Acrylates/ | 0.2 |
| | Polymer⁽¹⁾ | C10-30 Alkyl Acrylate | |
| | | Crosspolymer | |
| | Xanthan Gum PC⁽²⁾ | Xanthan Gum | 0.2 |
| B | Solution according to | Water (and) Capryloyl Glycine | 3.0 |
| | Example 1 | (and) Sodium Bicarbonate | |
| | 25% Caustic Soda | Water (and) Sodium Hydroxide | 0.3 |
| C | Glycerin | Glycerin | 20.0 |
| | Pentiol Green+⁽³⁾ | Pentylene Glycol | 3.0 |
| | Schercemol LL⁽¹⁾ | Lauryl Lactate | 1.0 |
| D | Perfume Karine⁽⁴⁾ | Perfume | 0.2 |
| | Citric Acid (50%) | Aqua (and) Citric Acid | pH 5.5 |
| | Buffer | Aqua (and) Citric Acid | 0.5 |
| | | (and) Sodium Citrate | |

Raw material suppliers:
⁽¹⁾Lubrizol,
⁽²⁾Jungbunzlauer,
⁽³⁾Minasolve,
⁽⁴⁾SFA

Carbopol is dispersed in water and stirred until completely hydrated. Xanthan gum is added, and the mixture is stirred until the solid is completely dispersed (Phase A). The solution according to the invention of Example 1 is added, followed by a little caustic soda (Phase B). The raw materials of Phase C are added, with stirring. Lastly, perfume is added, the pH is adjusted, and buffer solution is added. The product fulfills the A criteria of a preservative efficacy test according to ISO 11930.

### Example 5—Skin-Moisturizing Oil-in-Water Emulsion Lotion

| Phase | Raw Material | INCI Name | % |
|---|---|---|---|
| A | Water | Aqua | 77.4 |
| | Pentiol Green+⁽¹⁾ | Pentylene Glycol | 2.0 |
| | Xanthan Gum PC⁽²⁾ | Xanthan Gum | 0.5 |
| B | Solution according to | Water (and) Capryloyl | 6.0 |
| | Example 1 | Glycine (and) Sodium | |
| | | Bicarbonate | |
| C | Emulgade PL 68/50⁽³⁾ | Cetearyl Glucoside (and) | 5.0 |
| | | Cetearyl Alcohol | |
| | Lipex Sheasoft⁽⁴⁾ | Butyrospermum Parkii | 3.0 |
| | | (Shea) Butter | |
| | Jojoba Oil⁽⁵⁾ | Simmondsia Chinensis | 3.0 |
| | | (Jojoba) Seed Oil | |
| | Hazel Seed Oil⁽⁵⁾ | Corylus Avellana | 3.0 |
| | | (Hazel) Seed Oil | |
| D | DL-α-Tocopherol (>97%) | Tocopherol | 0.1 |
| E | Citric Acid (25%) | Aqua (and) Citric Acid | pH 5.5 |

Raw material suppliers:
⁽¹⁾Minasolve,
⁽²⁾Jungbunzlauer,
⁽³⁾BASF,
⁽⁴⁾AAK,
⁽⁵⁾Caesar & Loretz

Xanthan gum is mixed with Pentiol Green+ and then introduced into water, with stirring. The mixture is stirred until it becomes homogeneous (Phase A). The solution according to the invention (Phase B) is mixed in, and the mixture is heated to 75-80° C. Phase C is mixed and heated to 75-80° C. in a separate vessel. Phase C is added to Phase A+B while mixing with an Ultra-Turrax homogenizer. The emulsion is stirred at a high shear rate for 3 min and then for another 30 min with a propeller stirrer. The stirrer speed is reduced during cooling. Tocopherol (Phase D) is added at T<40 ° C. The pH is adjusted at room temperature (Phase E). The product fulfills the A criteria of a preservative efficacy test according to ISO 11930.

### Comparison Example 6—Skin-Moisturizing Oil-in-Water Emulsion Lotion, Produced with Solid Capryloyl Glycine

| Phase | Raw Material | INCI Name | % |
|---|---|---|---|
| A | Water | Aqua | 60.6 |
| | Pentiol Green+⁽¹⁾ | Pentylene Glycol | 2.0 |
| | Xanthan Gum PC⁽²⁾ | Xanthan Gum | 0.5 |
| B | Water | Aqua | 20.0 |
| | Sodium Hydroxide (50%) | Aqua (and) Sodium | 0.8 |
| | | Hydroxide | |
| | Caprocine⁽¹⁾ | Capryloyl Glycine | 2.0 |
| C | Emulgade PL 68/50⁽³⁾ | Cetearyl Glucoside (and) | 5.0 |
| | | Cetearyl Alcohol | |
| | Lipex Sheasoft⁽⁴⁾ | Butyrospermum Parkii | 3.0 |
| | | (Shea) Butter | |
| | Jojoba Oil⁽⁵⁾ | Simmondsia Chinensis | 3.0 |
| | | (Jojoba) Seed Oil | |
| | Hazel Seed Oil⁽⁵⁾ | Corylus Avellana | 3.0 |
| | | (Hazel) Seed Oil | |
| D | DL-α-Tocopherol (>97%) | Tocopherol | 0.1 |
| E | Citric Acid (25%) | Aqua (and) Citric Acid | pH 5.5 |

Raw material suppliers:
⁽¹⁾Minasolve,
⁽²⁾Jungbunzlauer,
⁽³⁾BASF,
⁽⁴⁾AAK,
⁽⁵⁾Caesar & Loretz

Xanthan gum is mixed with Pentiol Green+ and then introduced into water, with stirring. The mixture is stirred until it becomes homogeneous (Phase A). DI water is provided and mixed with aqueous caustic soda in a separate boiler. Solid capryloyl glycine is then added, with stirring. The mixture is stirred until a clear solution is formed (Phase B). Phase B is dispensed into Phase A and the mixture is then heated to 75-80° C. Phase C is mixed and heated to 75-80° C. in a third separate vessel. Phase C is added to Phase A+B while mixing with an Ultra-Turrax homogenizer. The emulsion is stirred at a high shear rate for 3 min and then for another 30 min with a propeller stirrer. The stirrer speed is reduced during cooling. Tocopherol (Phase D) is added at T<40 ° C. The pH is adjusted at room temperature (Phase E). The product fulfills the A criteria of a preservative efficacy test according to ISO 11930.

1. A method for producing an aqueous solution of an alkaline metal or alkaline earth metal salt of capryloyl glycinic acid and/or undecenoyl glycinic acid, comprising the following steps:
(i) Condensing a carboxylic acid selected from the group consisting of 1-octanoic acid (caprylic acid) and undecenoic acid in the form of an acid halide, a mixed anhydride or a symmetrical anhydride with glycine resulting in aqueous solution of a free N-acyl glycine,
(ii) Acidifying the solution thus obtained and extraction of the free N-acyl glycine in an organic solvent that is not completely miscible with water,
(iii) Conversion of the N-acyl glycine to an aqueous solution, with addition of a base, and
(iv) Removing impurities that are volatile with steam from the aqueous solution by azeotropic distillation of water.

2. The method according to claim 1, wherein the organic solvent used forms an azeotrope with water.

3. The method according to claim 1 wherein the capryloyl glycinic acid or undecenoyl glycinic acid is transferred, as an alkaline metal or alkaline earth metal salt, to an aqueous solution.

4. The method according to claim 1 wherein the capryloyl glycinic acid or undecenoyl glycinic acid is transferred, as a sodium and/or potassium salt, to an aqueous solution.

5. The method according to claim 1 wherein the free caprylic acid or undecenoic acid is removed from the aqueous capryloyl glycinic acid or undecenoyl glycinic acid solution by steam distillation at pH<8.

6. The method according to claim 1 wherein the aqueous solution thus obtained contains 10-50 wt % of the respective salt of capryloyl glycinic acid or undecenoyl glycinic acid.

7. The method according to claim 1 wherein the aqueous solution thus obtained does not contain any more than 2 wt % free caprylic acid or undecenoic acid.

8. The method according to claim 1 wherein the aqueous solution thus obtained does not contain more than 1 wt % of inorganic salts.

9. The method according to claim 6 wherein the aqueous solution thus obtained is incorporated into a cosmetic or dermatological product.

10. The use of a solution obtained according to claim 6 for inhibiting the growth or for killing of microorganisms.

11. The use of a solution obtained according to claim 6 for preserving cosmetic or dermatological products.
